(19) United States
(12) Patent Application Publication
(10) Pub. No.: US 2009/0012587 A1
(43) Pub. Date: Jan. 8, 2009

(54) MEDICAL LASER APPARATUS WITH ENHANCED DISINFECTION FUNCTION

(75) Inventors: SEAN XIAOLU WANG, WILMINGTON, DE (US); BRIAN PRYOR, EWING, NJ (US); QUN LI, Newark, DE (US)

Correspondence Address: FRANK F. TIAN, 331-4A THIRD AVENUE, LONG BEACH, NJ, 07740, US

(73) Assignee: BWT PROPERTY, INC., Newark, DE (US)

(21) Appl. No.: 12/165,461

(22) Filed: Jun. 30, 2008

Related U.S. Application Data
(60) Provisional application No. 60/947,689, filed on Jul. 3, 2007.

(51) Int. Cl. A61N 5/067; A61B 18/18

(52) U.S. Cl. 607/89; 606/11

(57) ABSTRACT

A laser apparatus is provided for medical applications or procedures, which include laser surgery, photo dynamic therapy, photo bio-modulation, etc. The output wavelength of the laser matches with the absorption band of oxygen molecule to produce highly reactive singlet oxygen from air or an oxygen enriched environment. The singlet oxygen is used as a strong oxidizer to kill bacteria. The properties of the laser such as power intensity, beam divergence, spectral linewidth, etc. are optimized for effective photo treatment of biological tissue as well as for efficient singlet oxygen generation.

### REFERENCE TO RELATED APPLICATIONS

This application claims an invention which was disclosed in Provisional Application No. 60/947,689, filed Jul. 3, 2007 entitled “MEDICAL LASER APPARATUS WITH ENHANCED DISINFECTION FUNCTION”. The benefit under 35 USC § 119(e) of the United States provisional application is hereby claimed, and the aforementioned application is hereby incorporated herein by reference.

### FIELD OF THE INVENTION

This invention generally relates to a laser apparatus, and more specifically to a medical laser apparatus with enhanced disinfection function.

### BACKGROUND

Medical lasers are widely used as surgical and therapeutic tools. One feature of the medical lasers is that they provide certain extent of disinfection function by killing bacteria through photo-thermal effect. However, the photo-thermal effect is a localized effect that occurs only at regions where the laser intensity is relatively high. Thus this thermal induced disinfection is inadequate for many medical applications, where other bactericidal methods have to be used for disinfection purposes. In addition, the photo-thermal effect may damage healthy tissue as well, which is not desirable for some photo therapy applications. There thus exists a need for an improved medical laser apparatus which provides enhanced disinfection function and induces minimal to no damage to healthy tissues.

### SUMMARY OF THE INVENTION

According to one aspect of the present invention, a laser apparatus is provided for medical applications or procedures, which include laser surgery, photo dynamic therapy, photo bio-modulation, etc. The output wavelength of the laser matches with the absorption band of oxygen molecules to produce highly reactive singlet oxygen from air or oxygen enriched environment. The singlet oxygen is used as a strong oxidizer to kill bacteria.

According to another aspect of the present invention, the properties of the laser such as power intensity, beam divergence, spectral linewidth, etc. are optimized for effective photo treatment of biological tissue as well as for efficient singlet oxygen generation.

### BRIEF DESCRIPTION OF THE FIGURES

The accompanying figures, where like reference numerals refer to identical or functionally similar elements throughout the separate views and which together with the detailed description below are incorporated in and form part of the specification, serve to further illustrate various embodiments and to explain various principles and advantages all in accordance with the present invention.

FIG. 1 illustrates one preferred embodiment of the present invention, where a fiber coupled medical laser with enhanced disinfection function is used for treatment of periodontal disease.

FIG. 2 illustrates another preferred embodiment of the present invention, where a diode based medical laser with enhanced disinfection function is used for photo bio-modulation.

Skilled artisans will appreciate that elements in the figures are illustrated for simplicity and clarity and have not necessarily been drawn to scale. For example, the dimensions of some of the elements in the figures may be exaggerated relative to other elements to help to improve understanding of embodiments of the present invention.

### DETAILED DESCRIPTION

Before describing in detail embodiments that are in accordance with the present invention, it should be observed that the embodiments reside primarily in combinations of method steps and apparatus components related to a medical laser apparatus with enhanced disinfection function. Accordingly, the apparatus components and method steps have been represented where appropriate by conventional symbols in the drawings, showing only those specific details that are pertinent to understanding the embodiments of the present invention so as not to obscure the disclosure with details that will be readily apparent to those of ordinary skill in the art having the benefit of the description herein.

In this document, relational terms such as first and second, top and bottom, and the like may be used solely to distinguish one entity or action from another entity or action without necessarily requiring or implying any actual such relationship or order between such entities or actions. The terms “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus. An element proceeded by “comprises . . . a” does not, without more constraints, preclude the existence of additional identical elements in the process, method, article, or apparatus that comprises the element.

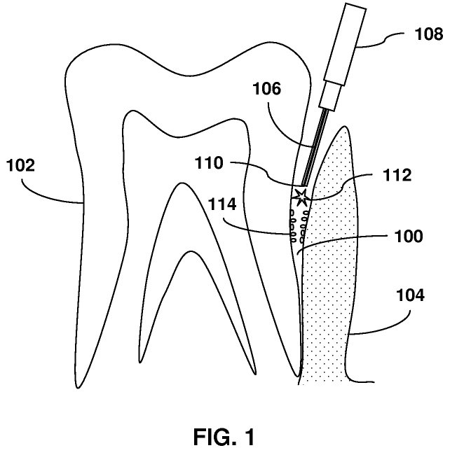

In the first preferred embodiment of the present invention, a fiber coupled medical laser with enhanced disinfection function is used for treatment of periodontal disease, which is caused by certain types of bacteria in the plaque. Referring to FIG. 1, the light from the medical laser (not shown) is delivered through an optical fiber 106 to the gingival pocket 100 formed between the tooth 102 and the gum 104. The optical fiber 106 is further mounted in a wand 108 for ease of handling by the operator. The optical fiber 106 is preferably designed with a small core diameter and a large numerical aperture so that very high light intensity will be produced near the tip 110 of the optical fiber 106. The wavelength of the medical laser falls within the absorption band of water and hemoglobin to be efficiently absorbed by the tissue for ablating and vaporizing purposes. In the mean time, the wavelength of the medical laser is selected and precisely controlled to match with one of the absorption band of oxygen molecule in the air to produce highly reactive singlet oxygen 112 in the vicinity of the fiber tip 110. In the present embodiment, the laser wavelength is preferably selected at about 764 nm or 1060 nm. The spectral linewidth of the laser is narrowed down to a value comparable to the absorption bandwidth of the oxygen molecule so that the laser energy can be efficiently absorbed by the oxygen molecule to pump the same to the excited state to produce singlet oxygen. In addition, the laser is stabilized to avoid any wavelength drift. During the treatment, the laser beam ablates and vaporizes the surface tissue of the gum 104 in the gingival pocket 100 and in the meantime produces singlet oxygen in the vicinity of the fiber tip 110, which acts as a broad spectrum antibacterial agent to kill bacteria 114 in the gingival pocket 100. After laser treatment, the gum 104 may be pressed toward the tooth 100 to restore adhesion. In a slight variation of the present embodiment, the medical laser may operate in a pulsed mode to further increase its peak power for more efficient singlet oxygen generation. In the meantime, the average laser power can be reduced to avoid excessive tissue damage.

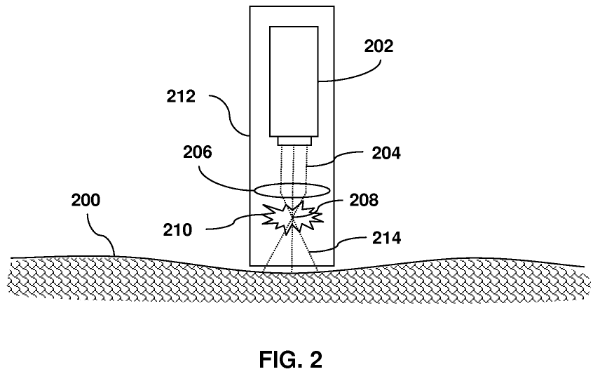

In the second preferred embodiment of the present invention, a diode based medical laser with enhanced disinfection function is used for photo bio-modulation. Referring to FIG. 2, the laser beam 204 from a diode laser 202 is focused by a lens 206 to produce concentrated hence higher light intensity at the focal point 208 of the lens 206. The laser wavelength is preferably selected at about 634 nm corresponding to one of the oxygen absorption peak to excite singlet oxygen 210 in the vicinity of the focal point 208 from oxygen molecules in the ambient air proximate to the focal point 208. Yet it is further preferred that the wavelength and spectral linewidth of the diode laser 202 are precisely controlled and narrowed down for more effective singlet oxygen generation. The singlet oxygen 210 acts as a broad spectrum antibacterial agent to kill bacteria on the surface of the target tissue 200. The spread laser beam 214, after the focal point 208, is employed to provide photo bio-modulation to the target tissue 200 for such purposes as: wound healing, pain relief, hair growth, etc. In the present embodiment, the lens 206 preferably has a small focal length so that high laser intensity can be produced around the focal point 208. In the meantime, the laser beam diverges rapidly after the focal point 208 and the laser intensity reduces correspondingly to avoid any thermal damage to the target tissue 200. The intensity of the laser beam on the target tissue 200 can be controlled by controlling the distance between the focal point 208 and the target tissue 200 through a laser holder 212.

In both of the two embodiments disclosed above, a flow of oxygen may be supplied proximate to a high intensity point of the laser beam, such as the fiber tip 110 shown in FIG. 1 or the focal point 208 shown in FIG. 2, to further improved the efficiency for singlet oxygen generation. The laser employed is not limited to the above listed types. Any kind of gas laser, fiber laser, upconversion fiber laser, diode laser, and diode pumped solid state laser with proper wavelength may be used.

In the foregoing specification, specific embodiments of the present invention have been described. However, one of ordinary skill in the art appreciates that various modifications and changes can be made without departing from the scope of the present invention as set forth in the claims below. Accordingly, the specification and figures are to be regarded in an illustrative rather than a restrictive sense, and all such modifications are intended to be included within the scope of present invention. The benefits, advantages, solutions to problems, and any element(s) that may cause any benefit, advantage, or solution to occur or become more pronounced are not to be construed as a critical, required, or essential features or elements of any or all the claims. The invention is defined solely by the appended claims including any amendments made during the pendency of this application and all equivalents of those claims as issued.

What is claimed is:

1. A method of making a device for providing photo treatment to subject biological tissue, the method comprising the steps of:
providing a laser unit to produce a laser beam having a wavelength falling within the absorption band of the subject biological tissue and in the meantime matches with an absorption band of oxygen molecules;
causing the laser beam to interact with the oxygen molecules naturally in the air or in an oxygen enriched environment to produce singlet oxygen and using said singlet oxygen to kill bacteria on or near the subject biological tissue; and
causing the laser beam to interact with the biological tissue for photo treatment.

2. The method of claim 1, wherein the photo treatment includes ablating, vaporizing, photocoagulation, photo bio-modulation, or photo bio-stimulation.

3. The method of claim 1, wherein the spectral linewidth of the laser unit is narrowed to match with the absorption bandwidth of the oxygen molecule.

4. The method of claim 1, wherein the laser unit is stabilized to avoid wavelength drift.

5. The method of claim 1, wherein the laser unit operates in a pulsed mode to increase the peak power of the laser beam.

6. The method of claim 1, wherein the laser beam is controlled to produce high laser intensity in a specific physical region for efficient singlet oxygen generation.

7. The method of claim 1, wherein a flow of oxygen is supplied proximate to a high intensity point of the laser beam.
